(19) United States
(12) Patent Application Publication
(10) Pub. No.: US 2024/0189504 A1
(43) Pub. Date: Jun. 13, 2024

(54) INSULIN PUMP WITH INTEGRATED CONTINUOUS GLUCOSE MONITOR

(71) Applicants: Michael Michaud, San Diego, CA (US); Philip Sven Lamb, San Diego, CA (US)

(72) Inventors: Michael Michaud, San Diego, CA (US); Philip Sven Lamb, San Diego, CA (US)

(21) Appl. No.: 18/220,156

(22) Filed: Jul. 10, 2023

Related U.S. Application Data
(60) Provisional application No. 63/359,759, filed on Jul. 8, 2022.

(51) Int. Cl. A61M 5/142; A61M 5/172; G16H 20/17

(52) U.S. Cl. CPC A61M 5/14244; A61M 5/1723; G16H 20/17; A61M 2230/201

(57) ABSTRACT

Embodiments disclosed herein are directed to ambulatory infusion pump systems that integrate a CGM sensor with an ambulatory infusion pump. An electrical connection can be established between the CGM sensor and the pump such that no wireless connectivity between the devices is required.

### RELATED APPLICATION

This application claims the benefit of U.S. Provisional Application No. 63/359,759 filed Jul. 8, 2022, which is hereby fully incorporated herein by reference.

### TECHNICAL FIELD

The present invention relates generally to ambulatory infusion pumps and, more particularly, to operation of ambulatory infusion pumps in conjunction with a continuous glucose monitor.

### BACKGROUND

There are a wide variety of medical treatments that include the administration of a therapeutic fluid in precise, known amounts at predetermined intervals. Devices and methods exist that are directed to the delivery of such fluids, which may be liquids or gases, are known in the art.

One category of such fluid delivery devices includes insulin injecting pumps developed for administering insulin to patients afflicted with type 1, or in some cases, type 2 diabetes. Some insulin injecting pumps are configured as portable or ambulatory infusion devices that can provide continuous subcutaneous insulin injection and/or infusion therapy as an alternative to multiple daily insulin injections via syringe or injector pen. Such ambulatory infusion pumps may be worn by the user, may use replaceable medicament cartridges, and may deliver other medicaments alone, or in combination with insulin. Such medicaments include glucagon, pramlintide, and the like. Examples of such pumps and various features associated therewith include those disclosed in U.S. Patent Publication Nos. 2013/0324928 and 2013/0053816 and U.S. Pat. Nos. 8,287,495; 8,573,027; 8,986,253; and 9,381,297, each of which is incorporated herein by reference in its entirety.

Ambulatory infusion pumps for delivering insulin or other medicaments can be used in conjunction with blood glucose monitoring systems, such as continuous glucose monitoring devices (CGMs). A CGM device consists of a sensor placed under the patient's skin and affixed to the patient via an adhesive patch, a transmitter, and a monitor. A CGM device samples the patient's interstitial fluid periodically (e.g., once every 1-5 minutes) to estimate blood glucose levels over time. CGMs are advantageous because they provide more frequent insights into a user's blood glucose levels yet do not require a finger stick each time a reading is taken.

Ambulatory infusion pumps may communicate with a dedicated CGM directly via a wired connection or indirectly via a wireless connection using wireless data communication protocols to communicate with a separate device (e.g., a dedicated remote device or a smartphone). One example of integration of ambulatory infusion pumps with CGM devices is described in U.S. Patent Publication No. 2014/0276419, which is hereby incorporated by reference herein. Ambulatory infusion pumps typically allow the user or caregiver to adjust the amount of insulin or other medicament delivered by a basal rate or a bolus, based on blood glucose data obtained by a CGM device, and in some cases include the capability to automatically adjust such medicament delivery. For example, based on CGM readings, some ambulatory infusion pumps may automatically adjust or prompt the user to adjust the level of medicament being administered or planned for administration or, in cases of abnormally low blood glucose readings, reducing or temporarily ceasing insulin administration.

As noted above, ambulatory infusion pumps often communicate wirelessly with a CGM. These communications therefore require a pairing procedure to establish communications between the devices. Both the pump and the CGM therefore also need to each have a battery, processor, communications element, etc.

### SUMMARY

Embodiments disclosed herein are directed to ambulatory infusion pump systems that integrate a CGM sensor with an ambulatory infusion pump. An electrical connection can be established between the CGM sensor and the pump such that no wireless connectivity between the devices is required.

In an embodiment, an ambulatory infusion pump includes a housing, a reservoir configured to contain a medicament, a pumping mechanism configured to deliver the medicament from the reservoir to a user and an electrical contact on the housing. The electrical contact can be configured to receive data from a glucose sensor. At least one processor can be configured to control the pumping mechanism to deliver the medicament from the reservoir to the user and to receive and process the data from the glucose sensor for use in calculating medicament delivery parameters.

In an embodiment, an ambulatory infusion pump system includes a glucose sensor configured to be inserted into a user and an ambulatory infusion pump. The ambulatory infusion pump can include a housing, a reservoir configured to contain a medicament, a pumping mechanism configured to deliver the medicament from the reservoir to a user and an electrical contact on the housing configured to receive data from the glucose sensor via an electrical connection. The pump can further include at least one processor configured to receive and process the data from the glucose sensor for use in calculating medicament delivery parameters.

In an embodiment, an ambulatory infusion pump system includes an ambulatory infusion pump including a housing, a reservoir configured to contain a medicament, a pumping mechanism configured to deliver the medicament from the reservoir to a user and an electrical contact on the housing configured to receive data from a glucose sensor via an electrical connection. The pump can further includes at least one processor configured to receive and process the data from the glucose sensor for use in calculating medicament delivery parameters. A tray can be configured to releasably hold the ambulatory infusion pump such that the tray facilitates the electrical connection between the ambulatory infusion pump and the glucose sensor.

The above summary is not intended to describe each illustrated embodiment or every implementation of the subject matter hereof. The figures and the detailed description that follow more particularly exemplify various embodiments.

### BRIEF DESCRIPTION OF THE DRAWINGS

Subject matter hereof may be more completely understood in consideration of the following detailed description of various embodiments in connection with the accompanying figures, in which:

FIG. 1 is an embodiment of an ambulatory infusion pump for use with embodiments of the disclosure.

FIG. 2 is a block of the ambulatory infusion pump of FIG. 1.

FIGS. 3A-3B are an alternate embodiment of an ambulatory infusion pump for use with embodiments of the disclosure.

FIG. 4 is an embodiment of a CGM for use with embodiments of the disclosure.

FIGS. 5A-5C depict an ambulatory infusion pump system according to the disclosure.

FIGS. 6A-6C depict an ambulatory infusion pump system according to the disclosure.

While various embodiments are amenable to various modifications and alternative forms, specifics thereof have been shown by way of example in the drawings and will be described in detail. It should be understood, however, that the intention is not to limit the claimed inventions to the particular embodiments described. On the contrary, the intention is to cover all modifications, equivalents, and alternatives falling within the spirit and scope of the subject matter as defined by the claims.

### DETAILED DESCRIPTION OF THE DRAWINGS

The following detailed description should be read with reference to the drawings in which similar elements in different drawings are numbered the same. The drawings, which are not necessarily to scale, depict illustrative embodiments and are not intended to limit the scope of the invention.

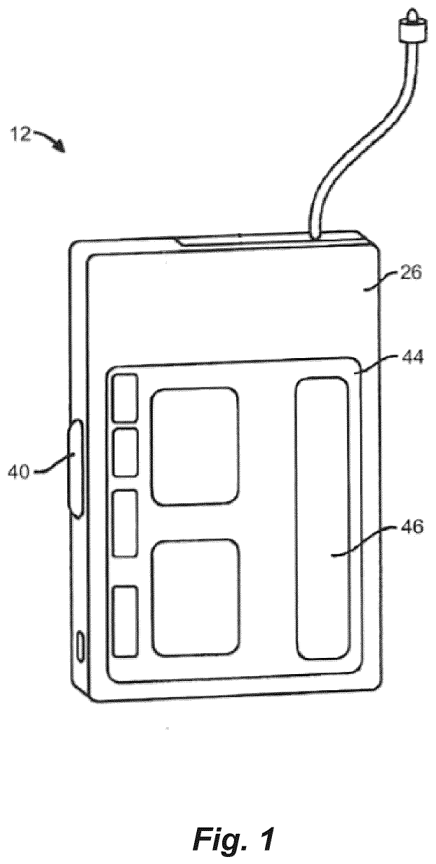

FIG. 1 depicts an example infusion pump that can be used in conjunction with one or more embodiments of the ambulatory infusion pump system of the present disclosure. Pump 12 includes a pumping or delivery mechanism and reservoir for delivering insulin or other medicament to a patient and an output/display 44. The output/display 44 may include an interactive and/or touch sensitive screen 46 having an input device such as, for example, a touch screen comprising a capacitive screen or a resistive screen. The pump 12 may additionally or instead include one or more of a keyboard, a microphone or other input devices known in the art for data entry, some or all of which may be separate from the display. The pump 12 may also include a capability to operatively couple to one or more other display devices such as a remote display (e.g., a dedicated remote display or a CGM display), a remote control device, or a consumer electronic device (e.g., laptop computer, personal computer, tablet computer, smartphone, electronic watch, electronic health or fitness monitor, or personal digital assistant). Further details regarding such pump devices can be found in U.S. Pat. No. 8,287,495, previously incorporated by reference above. It is to be appreciated that pump 12 may be optionally configured to deliver one or more additional or other medicaments to a patient.

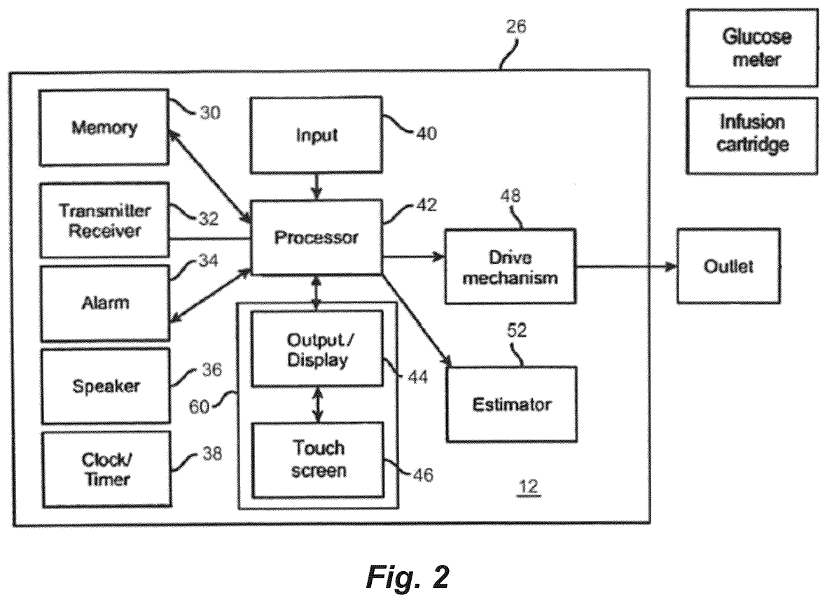

FIG. 2 illustrates a block diagram of some of the features that may be included within the housing 26 of pump 12. The pump 12 can include a processor 42 that controls the overall functions of the pump. The pump 12 may also include, e.g., a memory device 30, a transmitter/receiver 32, an alarm 34, a speaker 36, a clock/timer 38, an input device 40, a user interface suitable for accepting input and commands from a user such as a caregiver or patient, a drive mechanism 48, an estimator device 52 and a microphone (not pictured). One embodiment of a user interface is a graphical user interface (GUI) 60 having a touch sensitive screen 46 with input capability. In some embodiments, the processor 42 may communicate with one or more other processors within the pump 12 and/or one or more processors of other devices through the transmitter/receiver 32 such as a remote device (e.g., CGM device), a remote control device, or a consumer electronic device (e.g., laptop computer, personal computer, tablet computer, smartphone, electronic watch, electronic health or fitness monitor, or personal digital assistant). In some embodiments, the communication is effectuated wirelessly, by way of example only, via a near field communication (NFC) radio frequency (RF) transmitter or a transmitter operating according to a “Wi-Fi” or Bluetooth® protocol, Bluetooth® low energy protocol or the like. The processor 42 may also include programming to receive signals and/or other data from an input device, such as, by way of example, a pressure sensor, a temperature sensor, or the like.

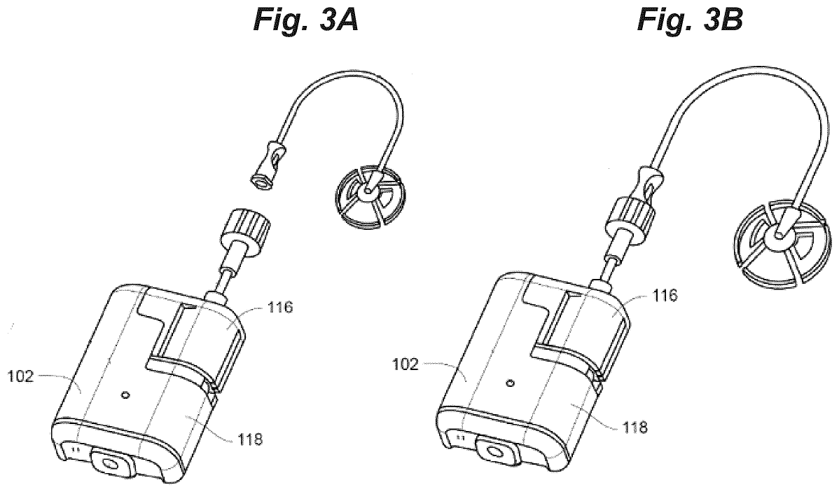

FIGS. 3A-3B depicts a second infusion pump that can be used in conjunction with one or more embodiments of the ambulatory infusion pump system of the present disclosure. Pump 102 includes a pump drive unit 118 and a medicament cartridge 116. Pump 102 includes a processor that may communicate with one or more processors within the pump 102 and/or one or more processors of other devices such as a remote device (e.g., a CGM device), a remote control device, or a consumer electronic device (e.g., laptop computer, personal computer, tablet computer, smartphone, electronic watch, electronic health or fitness monitor, or personal digital assistant). The processor 42 may also include programming to receive signals and/or other data from an input device, such as, by way of example, a pressure sensor, a temperature sensor, or the like. Pump 102 also includes a processor that controls some or all of the operations of the pump. In some embodiments, pump 102 receive commands from a separate device for control of some or all of the operations of the pump. Such separate device can include, for example, a dedicated remote control device or a consumer electronic device such as a smartphone executing an application configured to enable the device to transmit operating commands to the processor of pump 102. In some embodiments, processor can also transmit information to one or more separate devices, such as information pertaining to device parameters, alarms, reminders, pump status, etc. Such separate device can include any remote display, remote control device, or a consumer electronic device as described above. Pump 102 can also incorporate any or all of the features described with respect to pump 12 in FIG. 2. In some embodiments, the communication is effectuated wirelessly, by way of example only, via a near field communication (NFC) radio frequency (RF) transmitter or a transmitter operating according to a “Wi-Fi” or Bluetooth® protocol, Bluetooth® low energy protocol or the like. Further details regarding such pumps can be found in U.S. Pat. No. 10,279,106 and U.S. Patent Publication Nos. 2016/0339172 and 2017/0049957, each of which is hereby incorporated herein by reference in its entirety.

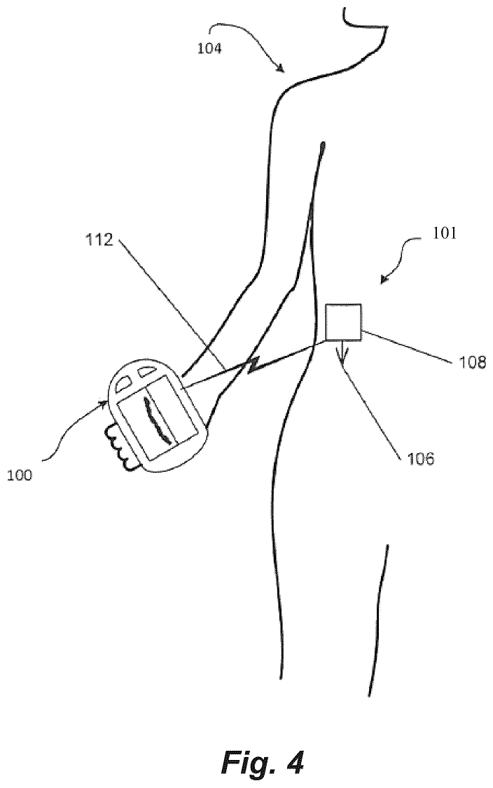

FIG. 4 depicts an example CGM system that can be used in conjunction with one or more embodiments of an ambulatory infusion pump system. The CGM system includes a sensor 101, a sensor probe 106, a sensor body 108, a receiver, and a monitor (receiver and monitor are depicted as device 100 in FIG. 4). The sensor 101 is removably affixed to a user 104 and includes a sensor probe 106 configured for transcutaneous insertion into the user 104. When placed, the sensor probe 106 reacts with the user's interstitial fluid which produces a signal that can be associated with the user's blood glucose level. The sensor 101 further includes a sensor body 108 that transmits data associated with the signal to the receiver 100 via wired or wireless connection (as represented by arrow line 112). In preferred embodiments, the receiver 100 receives the transmitted data wirelessly by any suitable means of wireless communication. By way of example only, this wireless communication may include a near field communication (NFC) radio frequency (RF) transmitter or a transmitter operating according to a “Wi-Fi” or Bluetooth® protocol, Bluetooth® low energy protocol or the like. Further detail regarding such systems and definitions of related terms can be found in, e.g., U.S. Pat. Nos. 8,311,749, 7,711,402 and 7,497,827, each of which is hereby incorporated by reference in its entirety.

Although as described above infusion pumps and continuous glucose monitors are separate devices, there are disadvantages to this configuration. For example, the requirement for Bluetooth or other wireless communications between the pump and the CGM require the devices to be paired. This requires the user to undertake a complex pairing procedure to pair the two devices and errors can arise if the devices become unpaired or communications issues arise between the devices. The CGM sensor and the pump also require two different attachment sites and separate insertions into the user's body. In addition, the separate devices each require their own batteries, processors, communications devices and associated circuitry. Embodiments described herein therefore incorporate the CGM into the ambulatory infusion pump to address each of these disadvantages.

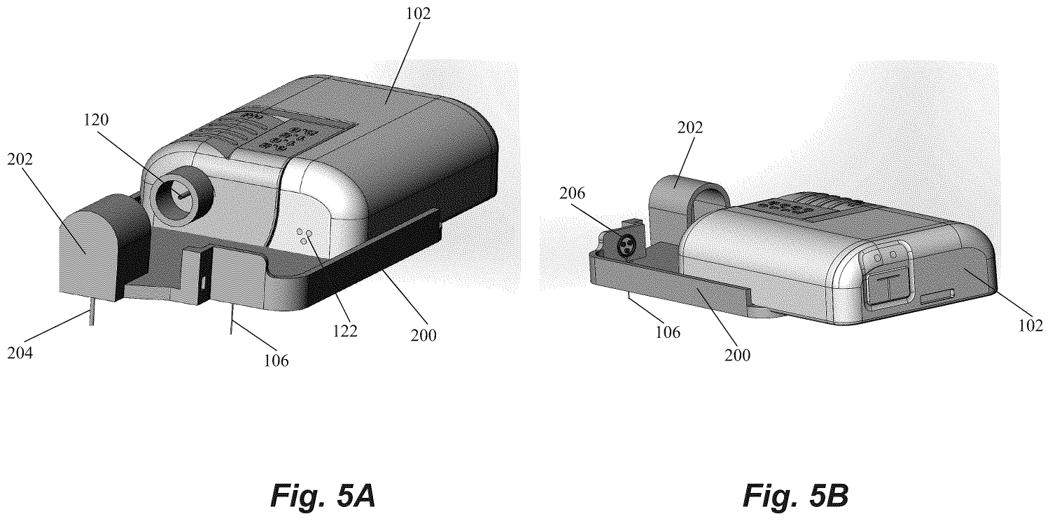

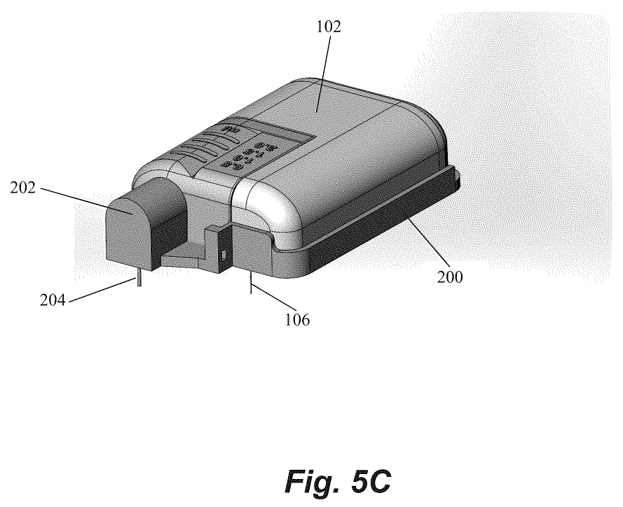

Referring to FIGS. 5A-5C, an ambulatory infusion pump system according to an embodiment includes a pump 102, a pump holder or tray 200 and a CGM sensor 106. CGM sensor 106 can be integrated into tray 200 such that sensor 106 extends through tray 200 and into the user's body when the user is wearing the system. Pump 102 can be retained on tray with a medicament outlet 120 of pump interfacing with a cannula hub 202 of tray to deliver medicament from pump to the user through a cannula 204 beneath tray. The cannula 204 for delivering medicament and the glucose sensor 106 can therefore both be integrated into tray 200 (though spaced apart in the depicted embodiment) such that the system requires only one attachment point on the body rather than separate attachment points. Glucose sensor 106 can be communicatively linked to sensor electrical connections 206 that are exposed on tray 200. Pump 102 can include corresponding electrical connections 122 exposed on an exterior surface of pump that interface with the sensor electrical connections 206 when the pump 102 is seated on the tray 200. This direct electrical connection can transfer glucose level data from the sensor 106 directly to the pump 102 to eliminate the need for wireless communications between the pump and a CGM. As can be seen in the figures both the medicament outlet 120 and the electrical connections 122 of the pump 102 can be interfaced with the corresponding cannula hub 202 and electrical connections 206 on the tray by sliding the pump 102 horizontally along the tray 200.

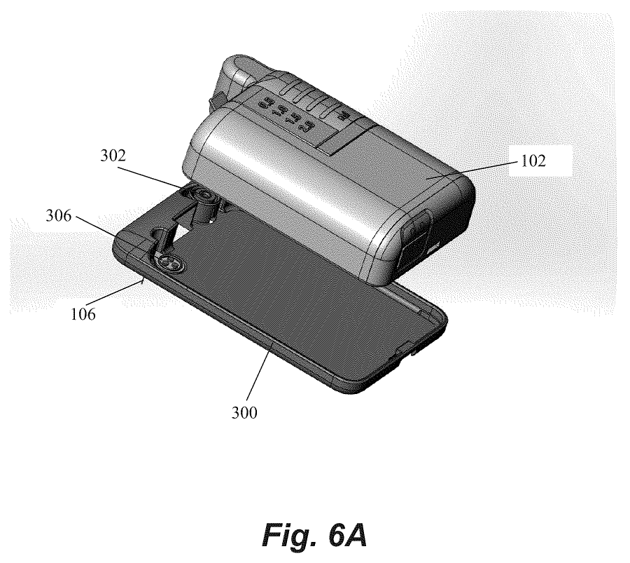

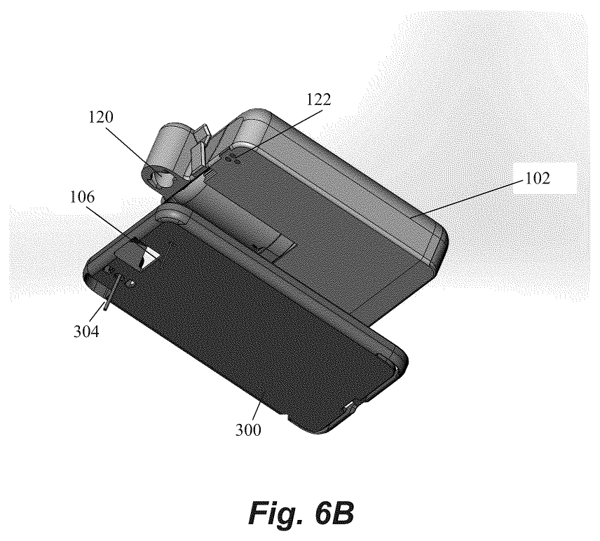

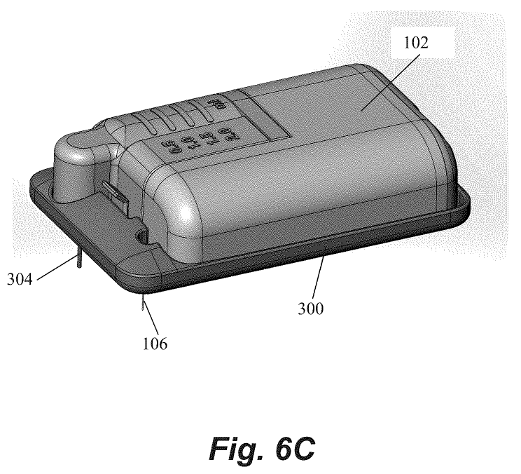

FIGS. 6A-6C depict an ambulatory infusion pump system according to another embodiment. This embodiment similarly includes a pump 102, a pump holder or tray 300 and glucose sensor 106. In this embodiment, the pump 102 is inserted vertically onto the tray 300. The electrical contacts 306 on the tray 300 coupled to the glucose sensor 106 are therefore on a bottom surface of the tray 300 and interface with corresponding electrical contacts 122 on a bottom surface of pump 102. Medicament outlet 120 on pump 102 also attaches vertically to cannula hub 302 on tray 300 to enable medicament to be delivered from pump 102 through a cannula 304 extending beneath tray 300.

In these embodiments, pump 102 includes internal circuitry to transmit the signals measured by the CGM sensor to the pump processor. The processor of pump can include programming sufficient to receive the CGM signals and to process those signals for use of the glucose levels indicated by the signals in providing therapy with the pump.

Integrating the CGM sensor with the pump 102 in the manner described herein addresses a number of issues with current systems. No Bluetooth or other wireless communications between the devices is required because the CGM sensor signals are sent to the pump via the direct electrical connection. As such, the CGM does not require a complex pairing procedure and wireless connectivity issues preventing transmission of CGM data will not occur. In addition, there are cost savings in the system because the CGM does not need a separate battery, processor, wireless communications elements and associated circuitry. Further, the disclosed integrated solutions require only one attachment site on the body rather than two separate placements with independent adhesive attachment points. The battery life of the pump will also be enhanced due to reduced power consumption from not having continual wireless communications with the CGM. In addition, the CGM does not require a separate battery as the pump battery can be used to provide the power required for the CGM sensor through for example, an inductive coupling, direct voltage, near field communications, etc.

The embodiments of FIGS. 5A-5C and 6A-6C depict embodiments in which the pump 102 is coupled directly to the CGM sensor 106 via electrical contacts interfaced through tray 200, 300. In another embodiment, the CGM sensor 106 can be positioned away from the tray by a relatively short distance, but still be directly coupled to the pump 102 via wire leads. This configuration similarly eliminates the need for wireless connectivity and communications between the CGM and the pump. In such an embodiment, the CGM sensor and infusion cannula need not be removed and/or replaced simultaneously such that the infusion cannula can be replaced more frequently than the CGM sensor.

It should be noted that while the embodiments described above primarily relate to an electrical contact that is exposed on the exterior of the housing of the pump to establish direct electrical connection with the glucose sensor, embodiments are contemplated in which the electrical contact is not exposed on the exterior of the housing and/or the connection is not a direct electrical connection. For example, the connection can be an indirect connection such as through a capacitive coupling or an inductive coupling.

In addition, although primary described with respect to a glucose sensor, it should be understood that integration of other types of analyte sensor in a same or similar manner is contemplated herein, alternatively or in addition to a glucose sensor.

In embodiments, an ambulatory infusion pump includes a housing, a reservoir configured to contain a medicament, a pumping mechanism configured to deliver the medicament from the reservoir to a user and an electrical contact on the housing. The electrical contact can be configured to receive data from a glucose sensor. At least one processor can be configured to control the pumping mechanism to deliver the medicament from the reservoir to the user and to receive and process the data from the glucose sensor for use in calculating medicament delivery parameters.

In some embodiments, the electrical contact is exposed on an exterior of the housing and is configured to receive data from the glucose sensor via a direct electrical connection.

In some embodiments, the electrical contact is configured to receive data from the glucose sensor via an indirect electrical connection.

In some embodiments, the electrical contact is configured to receive data from the glucose sensor via a capacitive coupling.

In some embodiments, the at least one processor is configured to receive the data from the glucose sensor without undergoing a pairing procedure that pairs the ambulatory infusion pump with the glucose sensor over wireless communications.

In some embodiments, the pump further comprises a battery, and the processor is configured to provide power to the glucose sensor with the battery.

In embodiments, an ambulatory infusion pump system includes a glucose sensor configured to be inserted into a user and an ambulatory infusion pump. The ambulatory infusion pump can include a housing, a reservoir configured to contain a medicament, a pumping mechanism configured to deliver the medicament from the reservoir to a user and an electrical contact on the housing configured to receive data from the glucose sensor via an electrical connection. The pump can further include at least one processor configured to receive and process the data from the glucose sensor for use in calculating medicament delivery parameters.

In some embodiments, the system further comprises a tray configured to releasably hold the ambulatory infusion pump, and the tray facilitates the electrical connection between the ambulatory infusion pump and the glucose sensor.

In some embodiments, the electrical contact is exposed on an exterior of the housing and is configured to receive data from the glucose sensor via a direct electrical connection.

In some embodiments, the electrical contact is configured to receive data from the glucose sensor via an indirect electrical connection.

In some embodiments, the electrical contact is configured to receive data from the glucose sensor via a capacitive coupling.

In some embodiments, the at least one processor is configured to receive the data from the glucose sensor without undergoing a pairing procedure that pairs the ambulatory infusion pump with the glucose sensor over wireless communications.

In some embodiments, the ambulatory infusion pump further comprises a battery, and the processor is configured to provide power to the glucose sensor with the battery.

In embodiments, an ambulatory infusion pump system includes an ambulatory infusion pump including a housing, a reservoir configured to contain a medicament, a pumping mechanism configured to deliver the medicament from the reservoir to a user and an electrical contact on the housing configured to receive data from a glucose sensor via an electrical connection. The pump can further includes at least one processor configured to receive and process the data from the glucose sensor for use in calculating medicament delivery parameters. A tray can be configured to releasably hold the ambulatory infusion pump such that the tray facilitates the electrical connection between the ambulatory infusion pump and the glucose sensor.

In some embodiments, the electrical contact is exposed on an exterior of the housing and is configured to receive data from the glucose sensor via a direct electrical connection through the tray.

In some embodiments, the electrical contact is configured to receive data from the glucose sensor via an indirect electrical connection.

In some embodiments, wherein the electrical contact is configured to receive data from the glucose sensor via a capacitive coupling.

In some embodiments, the at least one processor is configured to receive the data from the glucose sensor without undergoing a pairing procedure that pairs the ambulatory infusion pump with the glucose sensor over wireless communications.

In some embodiments, the ambulatory infusion pump further comprises a battery, and wherein the processor is configured to provide power to the glucose sensor with the battery.

Although embodiments described herein may be discussed in the context of the controlled delivery of insulin, delivery of other medicaments, singly or in combination with one another or with insulin, including, for example, glucagon, pramlintide, etc., as well as other applications are also contemplated. Device and method embodiments discussed herein may be used for pain medication, chemotherapy, iron chelation, immunoglobulin treatment, dextrose or saline IV delivery, treatment of various conditions including, e.g., pulmonary hypertension, or any other suitable indication or application. Non-medical applications are also contemplated.

Various embodiments of systems, devices, and methods have been described herein. These embodiments are given only by way of example and are not intended to limit the scope of the claimed inventions. It should be appreciated, moreover, that the various features of the embodiments that have been described may be combined in various ways to produce numerous additional embodiments. Moreover, while various materials, dimensions, shapes, configurations and locations, etc. have been described for use with disclosed embodiments, others besides those disclosed may be utilized without exceeding the scope of the claimed inventions.

Persons of ordinary skill in the relevant arts will recognize that the subject matter hereof may comprise fewer features than illustrated in any individual embodiment described above. The embodiments described herein are not meant to be an exhaustive presentation of the ways in which the various features of the subject matter hereof may be combined. Accordingly, the embodiments are not mutually exclusive combinations of features; rather, the various embodiments can comprise a combination of different individual features selected from different individual embodiments, as understood by persons of ordinary skill in the art. Moreover, elements described with respect to one embodiment can be implemented in other embodiments even when not described in such embodiments unless otherwise noted.

The entirety of each patent, patent application, publication, and document referenced herein is hereby incorporated by reference. Citation of the above patents, patent applications, publications and documents is not an admission that any of the foregoing is pertinent prior art, nor does it constitute any admission as to the contents or date of these documents.

Also incorporated herein by reference in their entirety are commonly owned U.S. Pat. Nos. 6,999,854; 8,133,197; 8,287,495; 8,408,421 8,448,824; 8,573,027; 8,650,937; 8,986,523; 9,173,998; 9,180,242; 9,180,243; 9,238,100; 9,242,043; 9,335,910; 9,381,271; 9,421,329; 9,486,171; 9,486,571; 9,492,608; 9,503,526; 9,555,186; 9,565,718; 9,603,995; 9,669,160; 9,715,327; 9,737,656; 9,750,871; 9,867,937; 9,867,953; 9,940,441; 9,993,595; 10,016,561; 10,201,656; 10,279,105; 10,279,106; 10,279,107; 10,357,603; 10,357,606; 10,492,141; 10/541,987; 10,569,016; 10,736,037; 10,888,655; 10,994,077; 11,116,901; 11,224,693; 11,291,763; 11,305,057; 11,458,246; 11,464,908; and 11,654,236 and commonly owned U.S. Patent Publication Nos. 2009/0287180; 2012/0123230; 2013/0053816; 2014/0276423; 2014/0276569; 2014/0276570; 2018/0071454; 2019/0307952; 2020/0206420; 2020/0329433; 2020/0368430; 2020/0372995; 2021/0001044; 2021/0113766; 2021/0353857; 2022/0062553; 2022/0139522; 2022/0223250; 2022/0233772; 2022/0233773; 2022/0238201; 2022/0265927; 2023/0034408; 2022/0344017; 2022/0370708; 2022/0037465; 2023/0040677; 2023/0047034; 2023/0113545 and 2023/0113755 and commonly owned U.S. patent application Ser. Nos. 17/368,968; 17/896,492; 18/011,060; 18/071,814; 18/071,835; 18/075,029; 18/090,788 18/115,316; and Ser. No. 18/139,391.

1. An ambulatory infusion pump, comprising:
a housing;
a reservoir configured to contain a medicament;
a pumping mechanism configured to deliver the medicament from the reservoir to a user;
an exterior electrical contact on the housing, the electrical contact configured to receive data from a glucose sensor; and
at least one processor configured to control the pumping mechanism to deliver the medicament from the reservoir to the user, the at least one processor configured to receive and process the data from the glucose sensor for use in calculating medicament delivery parameters.

2. The ambulatory infusion pump of claim 1, wherein the electrical contact is exposed on an exterior of the housing and is configured to receive data from the glucose sensor via a direct electrical connection.

3. The ambulatory infusion pump of claim 1, wherein the electrical contact is configured to receive data from the glucose sensor via an indirect electrical connection.

4. The ambulatory infusion pump of claim 3, wherein the electrical contact is configured to receive data from the glucose sensor via a capacitive coupling.

5. The ambulatory infusion pump of claim 1, wherein the at least one processor is configured to receive the data from the glucose sensor without undergoing a pairing procedure that pairs the ambulatory infusion pump with the glucose sensor over wireless communications.

6. The ambulatory infusion pump of claim 1, further comprising a battery, and wherein the processor is configured to provide power to the glucose sensor with the battery.

7. An ambulatory infusion pump system, comprising:
a glucose sensor configured to be inserted into a user; and
an ambulatory infusion pump including a housing, a reservoir configured to contain a medicament, a pumping mechanism configured to deliver the medicament from the reservoir to a user and an electrical contact on the housing configured to receive data from the glucose sensor via an electrical connection, wherein the pump further includes at least one processor configured to receive and process the data from the glucose sensor for use in calculating medicament delivery parameters.

8. The ambulatory infusion pump system of claim 7, further comprising a tray configured to releasably hold the ambulatory infusion pump, wherein the tray facilitates the electrical connection between the ambulatory infusion pump and the glucose sensor.

9. The ambulatory infusion pump system of claim 7, wherein the electrical contact is exposed on an exterior of the housing and is configured to receive data from the glucose sensor via a direct electrical connection.

10. The ambulatory infusion pump system of claim 7, wherein the electrical contact is configured to receive data from the glucose sensor via an indirect electrical connection.

11. The ambulatory infusion pump system of claim 10, wherein the electrical contact is configured to receive data from the glucose sensor via a capacitive coupling.

12. The ambulatory infusion pump of claim 7, wherein the at least one processor is configured to receive the data from the glucose sensor without undergoing a pairing procedure that pairs the ambulatory infusion pump with the glucose sensor over wireless communications.

13. The ambulatory infusion pump system of claim 1, wherein the ambulatory infusion pump further comprises a battery, and wherein the processor is configured to provide power to the glucose sensor with the battery.

14. An ambulatory infusion pump system, comprising:
an ambulatory infusion pump including a housing, a reservoir configured to contain a medicament, a pumping mechanism configured to deliver the medicament from the reservoir to a user and an electrical contact on the housing configured to receive data from a glucose sensor via an electrical connection, wherein the pump further includes at least one processor configured to receive and process the data from the glucose sensor for use in calculating medicament delivery parameters; and
a tray configured to releasably hold the ambulatory infusion pump, wherein the tray facilitates the electrical connection between the ambulatory infusion pump and the glucose sensor.

15. The ambulatory infusion pump system of claim 14, wherein the electrical contact is exposed on an exterior of the housing and is configured to receive data from the glucose sensor via a direct electrical connection through the tray.

16. The ambulatory infusion pump system of claim 14, wherein the electrical contact is configured to receive data from the glucose sensor via an indirect electrical connection.

17. The ambulatory infusion pump system of claim 16, wherein the electrical contact is configured to receive data from the glucose sensor via a capacitive coupling.

18. The ambulatory infusion pump of claim 14, wherein the at least one processor is configured to receive the data from the glucose sensor without undergoing a pairing procedure that pairs the ambulatory infusion pump with the glucose sensor over wireless communications.

19. The ambulatory infusion pump system of claim 14, wherein the ambulatory infusion pump further comprises a battery, and wherein the processor is configured to provide power to the glucose sensor with the battery.
